(19) United States
(12) Patent Application Publication
(10) Pub. No.: US 2014/0113286 A1
(43) Pub. Date: Apr. 24, 2014

(54) Epigenomic Markers of Cancer Metastasis

(71) Applicants: Timothy Chan, New York, NY (US); Fang Fang, New York, NY (US); Sevin Turcan, New York, NY (US)

(72) Inventors: Timothy Chan, New York, NY (US); Fang Fang, New York, NY (US); Sevin Turcan, New York, NY (US)

(73) Assignee: SLOAN-KETTERING INSTITUTE FOR CANCER RESEARCH, New York, NY (US)

(21) Appl. No.: 13/997,100

(22) PCT Filed: Dec. 21, 2011
(86) PCT No.: PCT/US11/66549

§ 371 Date: Jan. 8, 2014

Related U.S. Application Data
(60) Provisional application No. 61/425,610, filed on Dec. 21, 2010.

(51) Int. Cl. C12Q 1/68

(52) U.S. Cl. CPC C12Q 1/6886
USPC 435/6.11

(57) ABSTRACT

Methods and kits for assessing risk of metastasis in a cancer patient identified as having breast cancer, colon cancer or glioma use analysis of a classifier for CpG island methylator phenotype.

### FIELD OF THE INVENTION

This application relates to epigenomic marker sets for assessing the risk of cancer metastasis, and to the use of such marker sets in methods and kits. The marker sets are particularly applicable to methods and kits for use in connection with human breast cancer, colon cancer and glioma.

### BACKGROUND OF THE INVENTION

Breast cancer is one of the most prevalent human malignancies and is a major cause of cancer-related morbidity and mortality. Invasive ductal carcinoma (IDC) of the breast is a phenotypically diverse disease, consisting of tumors with varying pathologic and molecular characteristics (3-5). The primary biological subtypes of IDC include estrogen receptor (ER) and progesterone receptor (PR)-positive tumors (luminal A and B) and tumors that are ER/PR-negative (basal-like and HER2-enriched). These molecular determinants have significant effects on metastatic behavior, response to therapy, and clinical outcome. For example, ER/PR-positive tumors are generally associated with better clinical prognosis while basal-like (ER/PR-negative and HER2-negative, triple-negative) tumors are associated with higher rates of metastasis and death (6-9). The genomic alterations—including both genetic and epigenetic aberrations—underlying these differing metastatic potentials are ill-defined.

Significant effort has been undertaken to more accurately define the molecular alterations underlying breast cancer. For example, it has been shown that hormone receptor (HR) status is prognostic for clinical outcome. Mutations in genes such as BRCA1, PTEN, and PIK3CA help promote breast cancer oncogenesis, and are enriched in specific subgroups of IDC (10-12). Genomewide sequencing surveys have been performed to identify the scope of mutations in breast cancers (13-15). These data demonstrate that there exists substantial biological heterogeneity between and within the ER/PR positive and negative subgroups for which the molecular foundations remain obscure (13). In addition, gene expression classifiers have been developed to help predict metastatic risk. (16-18). Despite their increasing use in the clinic, the genomic root causes of these transcriptome differences that underlie metastatic potential remains unclear.

In addition to genomic variations of this type, changes in phenotype or gene expression caused by mechanisms other than changes in the underlying DNA sequence may occur and be involved in the onset and progression of cancers. Changes of this type are referred to as “epigenetic” or “epigenomic” variations.

Widespread changes in DNA methylation patterns have been reported to occur during oncogenesis and tumor progression (19, 20). Cancer specific changes in DNA methylation can alter genetic stability, genomic structure, and gene expression (21, 22). Promoter CpG island methylation can result in transcriptional silencing, and thus loss of function of tumor suppressor genes, and plays an important role in the oncogenic process (19). CIMP (CpG island methylator phenotype), which is associated with a strong tendency to hypermethylate specific loci, has been described in a subset of colorectal cancers, and recently in a subgroup of gliomas (2, 23). Aberrations in DNA methylation have been reported in human breast cancer but the impact of the methylome on metastasis and the presence of breast CIMP has remained elusive (24-30).

US Patent Publication No. 2010/0273164, which is incorporated herein by reference, discloses methods for detection of methylated cytosine residues in a target nucleic acid. Van der Auwera et al., PLosOne (2010) 5: 1-10, which is incorporated herein by reference, discloses evaluation of methylation in breast cancer to arrive at a 14 gene classifier in which methylation of NIP, CHGA, OSR1, GFRA3, KLK10, SSTR1, EFCPB2, PPARG, PRKAR1B, ABCG2, FGF5, PLTP, GRASP and PAX7 is used to distinguish inflammatory breast cancer from non-inflammatory. Van der Auwere et al. also reported that high methylation with this classifier was observed in samples with distant metastases and poor prognosis. US Patent Publication No. 2010/0209906 which is incorporated herein by reference relates to detection of methylation in colon cancer.

### SUMMARY OF THE INVENTION

The present application provides a classifier that can be used in the prediction of metastatic risk in a patient, with particular applicability to patients diagnosed with breast cancer, colon cancer, or glioma.

In one aspect, the invention provides a method for assessing risk of metastasis in a cancer patient identified as having breast cancer, colon cancer or glioma, comprising the steps of:

(a) obtaining a sample of tumor tissue from the patient;

(b) evaluating the sample for hypermethylation of a plurality of genes, and

(c) based on the evaluation of step (b) determining whether and/or to what extent the patient is at risk of cancer metastasis. The plurality of genes includes at least three genes selected from the group consisting of: ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1. In exemplary embodiments, 3-20 genes, for example 3-10 genes, from this list are evaluated in a patient with breast cancer.

In specific embodiments, the plurality of genes includes at least three genes selected from among RASGF2, ARHGEF7, FBN1, SOX8, CRABP1, FOXL2, and ALX4.

In a further aspect of the invention, a set of 33 genes is provided as a single classifier that can be used in prediction of risk of metastasis in patients with breast cancer, colon cancer or glioma. This set of genes includes three genes from the set of genes above plus additional genes. Using this classifier, methylation is assessed for ADAM12, ALX4, FOX12, ACOT12, ACTA1, AOX1, C1orf76, CD8A, DES, DMN, DMT1, DYPSL4, EYA4, FLJ14834, GCM2, GSH2, LOC112937, LOC389112937, LOC399458, MAL, MEGF10, MGC26856, NEUROG1, PDPN, RPL39L, SFRP2, SLC13A5, SYT6, TFP12, THSD3, TLR2 and TP73.

A further aspect of the invention is a kit which may be used in methods for assessment of metastatic. Such a kit consists essentially of materials for the evaluation of metastasis risk in a cancer patient identified as having breast cancer, colon cancer or glioma, said kit includes reagents for determination of the extent of methylation of a plurality of genes, wherein the plurality of genes includes at least three genes selected from the group consisting of: ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1. In exemplary embodiments, the kit includes materials for assessment of less than 100 genes, preferably less than 50 genes. For example, the kit may include materials for assessing methylation in 3 to 20 genes, for example 3-10 genes, from the above list.

In another embodiment, the kit includes materials for assessing methylation in the genes ACOT12, ACTA1, AOX1, C1orf76, CD8A, DES, DMN, DMT1, DYPSL4, EYA4, FLJ14834, GCM2, GSH2, LOC112937, LOC389112937, LOC399458, MAL, MEGF10, MGC26856, NEUROG1, PDPN, RPL39L, SFRP2, SLC13A5, SYT6, TFP12, THSD3, TLR2 and TP73

### BRIEF DESCRIPTION OF THE DRAWINGS

FIG. 1A: Validation of B-CIMP loci methylation using EpiTYPER mass spectrometry. EpiTYPER was used to analyze the methylation state of the CpG islands of the genes indicated. Each circle indicates a CpG dinucleotide. The frequency of methylated alleles is shown by the color scale in the legend. The genomic location is noted. IVD, in vitro methylated DNA; WGA, whole genome amplified DNA; NB, normal breast.

FIGS. 1B and C: B-CIMP positive tumors are highly associated with hormone receptor (1B) positivity but not HER2 status (1C).

FIG. 1D: Relative methylation (normalized and transformed beta-value) of the genes analyzed in CIMP+ versus CIMP− tumors. P-value indicating significance determined using ANOVA.

FIG. 1E: Relative methylation of the genes analyzed in FIG. 1 in ER/PR-positive versus ER/PR-negative tumors. Significance determined using ANOVA.

FIG. 1F: Relative methylation of the genes analyzed in FIG. 1 in ER/PR-positive, CIMP+ versus ER/PR-positive, CIMP− tumors. Significance determined using ANOVA.

FIG. 1G: Kaplan-Meier (KM) curve for distant metastasis-free survival for B-CIMP-positive and B-CIMP-negative subtypes. Significance calculated by log-rank analysis. Data from discovery set tumors (FIG. 1).

FIG. 1H: Validation of B-CIMP and its impact on metastatic risk in an independent set of breast tumors (n=132). EpiTYPER assays were developed for three of the most predictive genes for B-CIMP as indicated on the left panel. Samples were analyzed and categorized as CIMP+ if at least 2 out of 3 genes were methylated. red=methylated, blue=unmethylated, purple=CIMP+. KM curves for distant metastasis-free survival (middle panel) and overall survival (right panel) by B-CIMP status. Significance calculated by log-rank analysis.

FIGS. 1 I-K: CIMP predicts metastatic risk in ER/PR+ breast cancers. Kaplan-Meier (KM) curve for distant metastasis-free survival for B-CIMP+ and B-CIMP− subtypes. Significance calculated by log-rank analysis.

FIG. 2: KM survival curve showing that the CIMP repression signature (hypermethylated and down-regulated in B-CIMP tumors) predicts for survival in the van't Veer cohort EN.CITEEN.CITE.DATA (17). P-value calculated by log-rank.

FIG. 3A: Venn diagram showing common targets between polycomb complex 2 (PcG2) targets described in EN.CITEEN.CITE.DATA (47) and CIMP in the three indicated cancers. Genes in parentheses indicate number of genes in common between PcG2 target genes and CIMP targets in each cancer type. The table below the diagram shows the level of significance between these overlapping gene lists (p-value, hypergeometric distribution). The numbers in the Venn diagram show the number of CIMP/PcG2 common targets that are shared between the cancer types.

FIG. 3B: Same as in FIG. 3A except polycomb targets are from the Suz12 targets described in EN.CITEEN.CITE.DATA (46).

### DETAILED DESCRIPTION OF THE INVENTION

The present invention is based on a genome-wide analysis to characterize the methylomes of breast cancers with diverse metastatic behavior. This analysis led to the identification of a subset of breast tumors that display coordinate hypermethylation at a large number of genes, demonstrating the existence of a breast-CpG island methylator phenotype (B-CIMP). B-CIMP imparts a distinct epigenomic profile and is a strong determinant of metastatic behavior. B-CIMP loci are highly enriched for genes that define metastatic potential. Importantly, methylation at B-CIMP genes account for much of the transcriptomal diversity between breast cancers of varying prognosis, indicating a fundamental epigenomic contribution to metastatic risk.

As used in this application, the term “risk of metastasis” refers to a prognostic indication that the cancer in a particular patient, particularly a human patient, will advance to a metastatic state based on statistical predictors. Actual advance to a metastatic state is not required, and adoption of treatment modalities to try to delay or prevent the realization of such risk is anticipated to occur.

As used in this application, the term “obtaining a sample of tumor tissue from the patient” refers to obtaining a specimen of tumor, for example a biopsy specimen, or a portion of a surgically excised specimen from a patient for use in testing. The sample may be collected by the person performing the assay procedures, but will more commonly be collected by a third party and then sent for assay. Either the actual collection or the receipt of a sample for assay is within the scope of the term “obtaining a sample.”

In the assay methods and kits of the invention, the sample is evaluated for the extent of methylation, and preferably for hypermethylation of a plurality of genes. In general the number of genes will be less than 50 genes, and preferably will be in the range of 3 to 20 genes, for preferably 3 to 10 genes. Selection of the genes and the number of genes evaluated is suitably based the prognostic value of the genes. Where genes with higher prognostic value are evaluated, fewer genes need to be evaluated to arrive at a reliable indication of risk of breast cancer metastasis. A gene with a high prognostic value is one that has a high correlation between hypermethylation and metastasis risk. For example, in Table 2, the q value in Column 4 is an indicator the statistical significance of the relation between hypermethylation of the indicated gene and a decrease in metastatic risk. It can be seen that ALX4, when analysed with the probeset cg04988423 has a very high statistical significance (small q value). Thus, tools that include analysis of this gene will need fewer tests to achieve statistical reliability. On the other hand, tests that include no genes from the top 50 genes in Table 2 should evaluate more genes in the assay method and/or kit.

In some embodiments, the kits of the present invention consist essentially of materials for the evaluation of metastasis risk in a cancer patient identified as having breast cancer, colon cancer or glioma and include materials for detection of the extent of methylation in at least some specified genes. As used in this context, the term “consisting essentially of” means that the kit does not include materials that provide functionality other than the evaluation of metastasis risk to any significant extent. In particular, the kit does not encompass a set of broad screening reagents such as found on an Affymetrix® chip or and Illumina Human Methylation27 beadarray, which may include the relevant genes in combination with a multitude of genes that are not relevant to metastasis prediction. The kit might, however, include materials for evaluation of some additional genes, provided these do not change the primary purpose of the kit.

Table 1 sets forth a subset of genes that have been found by the inventors to have prognostic value for prediction of metastasis risk in order of significance as well as suitable probe sets for each protein listed as Differentially methylated Probeset IDs from Illumina Human Methylation27 beadarray. These beadarrays query 27,578 CpG islands each, covering 14,495 genes.

In embodiments of the invention, the genes evaluated are selected from this list. In some embodiments, all of the genes in Table 1 are evaluated. In some embodiments, at least 50 genes in Table 1 are evaluated. In some embodiments, from 3 to 20 genes in Table 1 are evaluated. In specific embodiments, the plurality of genes includes at least three genes selected from among RASGF2, ARHGEF7, FBN1, SOX8, CRABP1, FOXL2, and ALX4. In some embodiments, the gene tested include the top 3, 5, 8 or 10 genes listed in Table 1.

Methylation of these genes may be tested in combination with other genes that have been shown to be of relevance in a other CIMP classifiers without departing from the scope of the invention.

Based on the evaluation results, whether and/or to what extent the patient is at risk of breast cancer metastasis is determined. It will be appreciated that the significance of hyper or hypomethylation to metastatic risk depends on the gene that is hyper or hypomethylated. For example, as discussed below, observation of hypermethylation in ALX4, ARHGEF7, and RASGRF2 correlated with a decreased incidence of metastatic relapse. This is the case for each of the genes in Table 1, and in Table 2.

On a practical level, risk is measured by detecting methylation in a subset of the CIMP genes. The genes that can be used can include any combination of our B-CIMP genes as described in Table 1 or Table 2. A panel of 3-10 genes detected using quantitative methylation specific PCR, EpiTYPER, or methyllight can be used in the clinic. Methylation of these genes determines whether the breast tumor is CIMP+ or −. This information is used in conjunction with standard staging and pathology to determine risk of metastasis. If risk is sufficiently high (determined on a case by case basis via clinical practice standards), then patient may be offered more aggressive chemotherapy.

Other methods for detection of methylation at the nucleic level are also known, and may be used in the methods of the invention. For example, as described in U.S. Pat. No. 7,153,653, which is incorporated herein by reference, mapping of methylated regions in DNA may be based on Southern hybridization approaches, based on the inability of methylation-sensitive restriction enzymes to cleave sequences which contain one or more methylated CpG sites, or using methylated CpG island amplification (MCA) to enrich for methylated CpG rich sequences. MCA coupled with Representation Difference Analysis (MCA/RDA) can recover CpG islands differentially methylated in cancer cells (Toyota, et al., Cancer Res. 59:2307 2312, 1997).

Because CpG island methylation leads to reduced expression of the gene and associated proteins, methylation can also be assessed indirectly through assessment of gene expression and expressed protein levels. This assays can be performed using an Affymetrix microarray, or immunohistochemistry. By way of example, if this approach is used, assessment of risk can be made on the basis of an assay for some combination of the 102 hypermethylated and down-regulated genes of Table 3. In most cases, however, methylation assays are preferred over expression-based assays since methylation assays are more robust, less expensive, and can be used on samples that are easier to obtain from the clinic, DNA being more stable than RNA.

To facilitate the performance of the methods of the invention as prognostic evaluations on actual patient samples, the present invention provides diagnostic assay tools/kits that include reagents sufficient to do the testing without the overhead of numerous additional and less relevant reagents that might be present in a research tool. Thus, in accordance with an embodiment of the invention, the assay kits of the invention comprise reagents for determination of CpG island methylation of 100 genes or less, preferably 50 genes or less, in which at least 50% of the genes for which reagents are provided are genes that have relevance to the determination of risk of breast cancer metastases.

In accordance with some embodiments, the kits of the invention contain reagents for detection of methylation in 3 to 20 genes. In specific embodiments, the plurality of genes includes at least three genes selected from among RASGF2, ARHGEF7, FBN1, SOX8, CRABP1, FOXL2, and ALX4. In some embodiments, the gene tested include at least the top 3, 5, 8 or 10 genes listed in Table 1, any three genes of the top 5, any three genes of the top 8 or any three genes of the top 10 genes listed in Table 1.

In some embodiments of the invention, the target specific reagents contained in the kit include reagents for detection of methylation of a gene set as discussed above. The specific nature of the reagent will depend on the methodology employed for determination of methylation, but may include sequence specific probes or primers. Optionally, the kit may also include the non-target-specific reagents. The reagents may be provided in an array format for ease of use and interpretation.

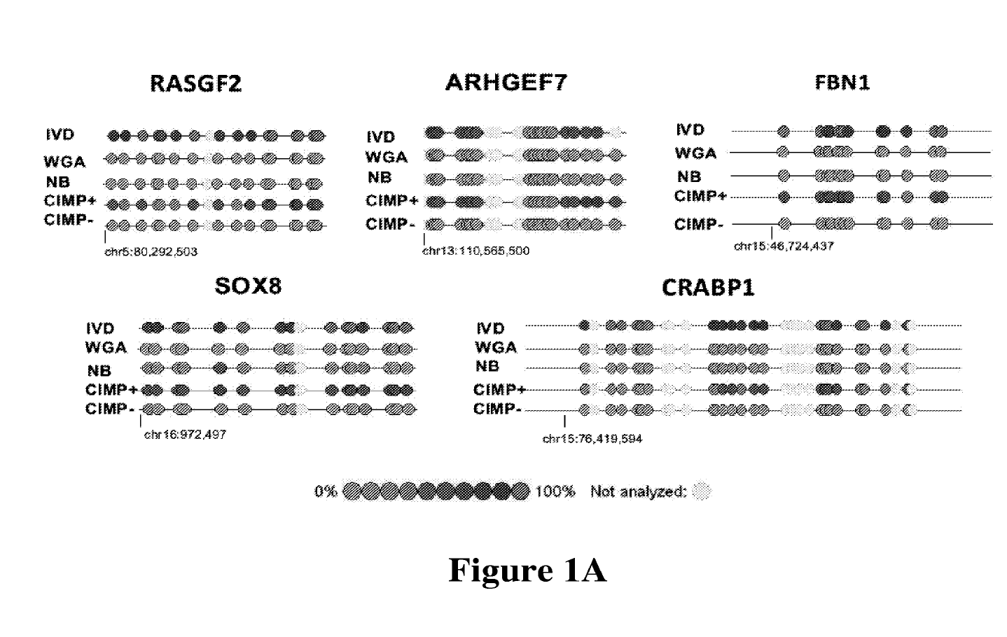

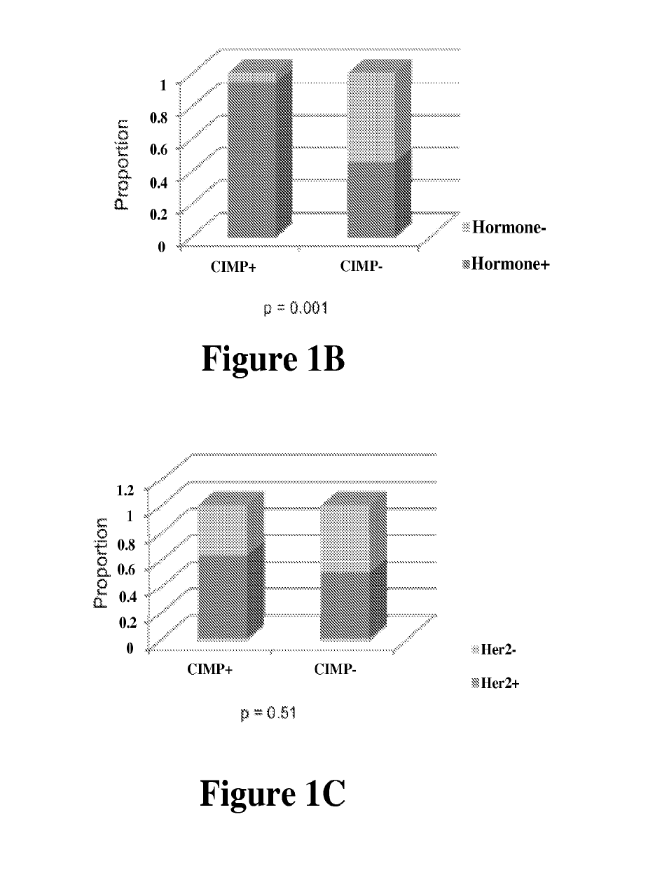

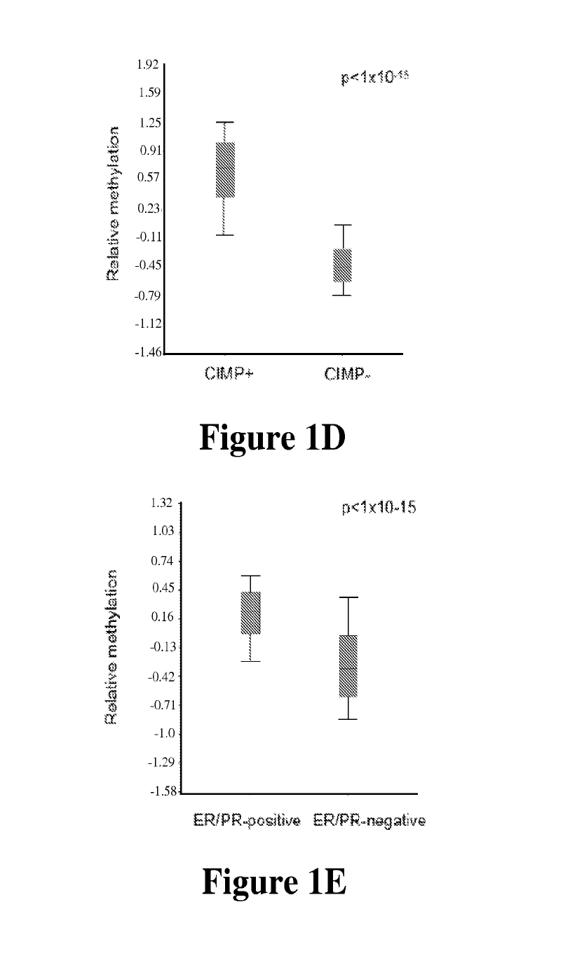

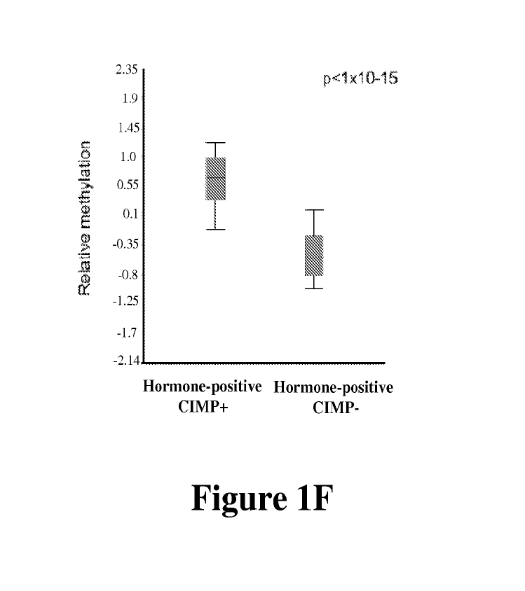

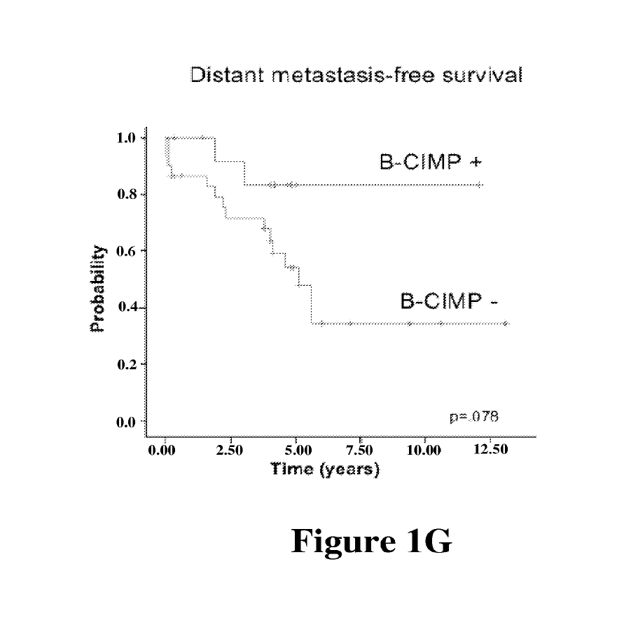

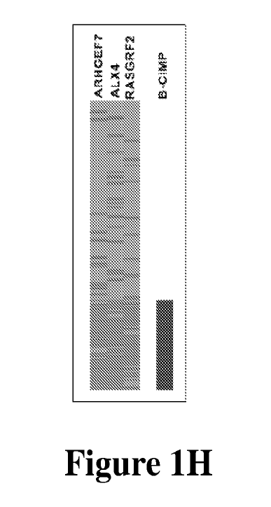

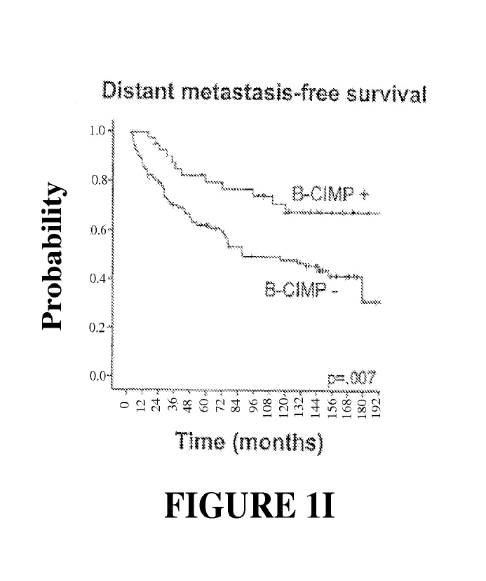

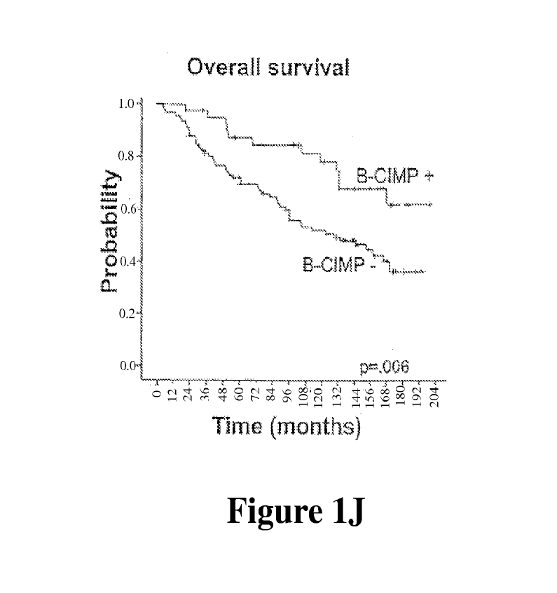

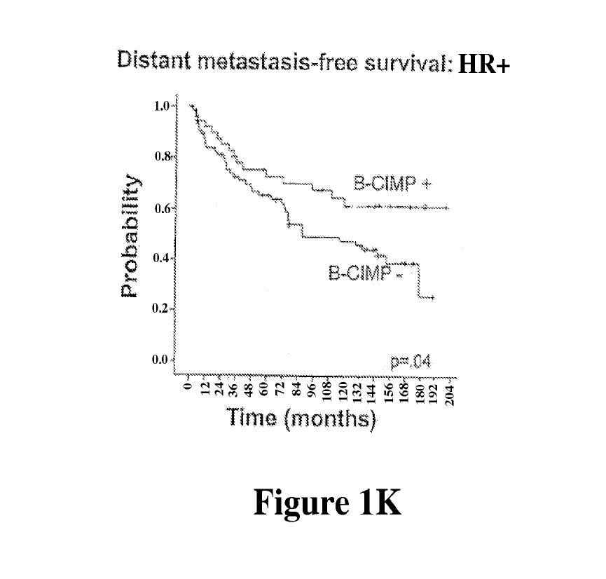

The results summarized above are derived from a systematic, genome-wide characterization of the breast cancer methylome in breast cancers with diverse metastatic behavior. We used the Illumina Infinium HumanMethylation27 platform because it provides efficient genome-wide interrogation of CpG islands. This platform is well-validated and highly reproducible (mean correlation coefficient=0.987) (31, 32). Using this platform, analyses of replicate breast cancer samples generated highly concordant data. We first analyzed a discovery set of IDCs with differing metastatic behavior (including samples with varying ER/PR and HER2 status from patients with excellent clinical followup (n=39, Table 4). To identify breast cancer subgroups, we selected the most variant probes and performed consensus clustering and unsupervised hierarchical clustering. We identified two robust DNA methylation clusters, one encompassing a portion of the HR-positive tumors (defined as ER/PR-positive, cluster 2) and one encompassing tumors that were ER/PR-positive or ER/PR-negative (cluster 1). Cluster 2 breast cancer samples possessed a highly characteristic DNA methylation profile with high coordinate hypermethylation at a subset of loci, similar to the CIMP phenotype seen in colorectal cancer (2, 23). We, thus, designated this group (cluster 2) as having a breast CpG island methylator phenotype (B-CIMP). In our discovery set, 17 out of 39 (44%) of tumors were B-CIMP-positive (B-CIMP+) Importantly, the composition of the B-CIMP+ subgroup was confirmed by two independent clustering algorithms (2D hierarchical and K-means consensus clustering); both approaches defined the same set of tumors as exhibiting B-CIMP. Cluster significance was evaluated by SigClust and class boundaries were highly significant (33). Our array results were validated using EpiTyper (34), a mass spectrometry-based technique allowing sensitive detection of DNA methylation at base-pair resolution (FIG. 1A). We observed strong concordance between B-CIMP calls on the Infinium platform and those from Epityper. These results demonstrate that there exist profound differences across the methylomes of breast cancers. While it is possible that additional, smaller subgroups exist, the robust nature of our consensus modeling here suggests that there are two primary epigenomic subgroups of IDC as defined by genome-wide methylome profiling.

We defined the relationship between B-CIMP status, clinical co-variates, and known molecular determinants. Interestingly, CIMP+ tumors consisted almost entirely of ER/PR-positive tumors (94%, FIG. 1B) (16/17) while CIMP− tumors consisted of similar numbers of HR-positive (45%)(10/22) and HR-negative tumors (55%)(12/22)(p=0.001, chi-squared test)(FIG. 1C). In contrast, there was no significant difference in frequency of HER2 positivity between the CIMP groups. Average methylation intensities were significantly different between CIMP+ versus CIMP−, ER/PR-positive versus ER/PR-negative, and HR-positive/CIMP+ versus HR-positive/CIMP− tumors (FIG. 1D-F). There was a strong trend towards improved distant metastasis-free survival in patients with CIMP+ tumors in our discovery set (FIG. 1G).

To validate the existence of B-CIMP, we used Epityper to evaluate an independent cohort of breast cancers. We examined methylation of three loci (ALX4, ARHGEF7, and RASGRF2) that were among the most predictive for B-CIMP in our Infinium data (FIG. 1H). In this set, B-CIMP+ tumors demonstrated a significantly lower risk for metastatic relapse and death (FIG. 1I-K). The presence of B-CIMP was an independent predictor for survival on multivariate analysis (p=0.06).

We next sought to define the nature of the methylome differences between the B-CIMP subgroups and characterize the effects of these differences on the breast cancer transcriptome. Probes were filtered for analysis by ranking transformed beta-values using decreasing adjusted p-values and increasing beta-value difference to identify the top most differentially hypermethylated genes in the B-CIMP group. Of the 3297 CpG sites that were differentially methylated between CIMP+ and CIMP− tumors, 2333 (71%) were hypermethylated (Tables 5-7). There were 2543 unique genes represented within this group, including 1764 that were hypermethylated and 779 that were hypomethylated (See Table 13).

Affymetrix transcriptome data were obtained from the same breast tumors analyzed for methylation to determine genes demonstrating differential expression and B-CIMP methylation. A total of 279 genes were significantly downregulated and 238 genes were significantly upregulated (Table 8). Gene ontology (GO) analysis showed that the significantly upregulated genes were highly enriched for functional categories involving cell motion, angiogenesis, apoptosis, development, kinase activity, and DNA binding (FIG. S4A-B). The downregulated genes were enriched for functional categories involved in mitosis, cytokinesis, exocytosis, chromosomal segregation, transcription factor activity, and kinase activity. Integration of the normalized gene expression and DNA methylation gene sets identified 102 genes with both significant hypermethylation and downregulation in B-CIMP-positive tumors (Table 3). Among these genes are LYN, MMP7, KLK10, and WNT6, which are known to play a role in breast cancer outcome or epithelial-mesenchymal transition (EMT) (35,36,37,38). GO analysis showed B-CIMP-specific downregulation of genes (hypermethylated and downregulated in B-CIMP) is associated with cell motion, development, signaling, and catalytic activity as some of the most significant functional categories.

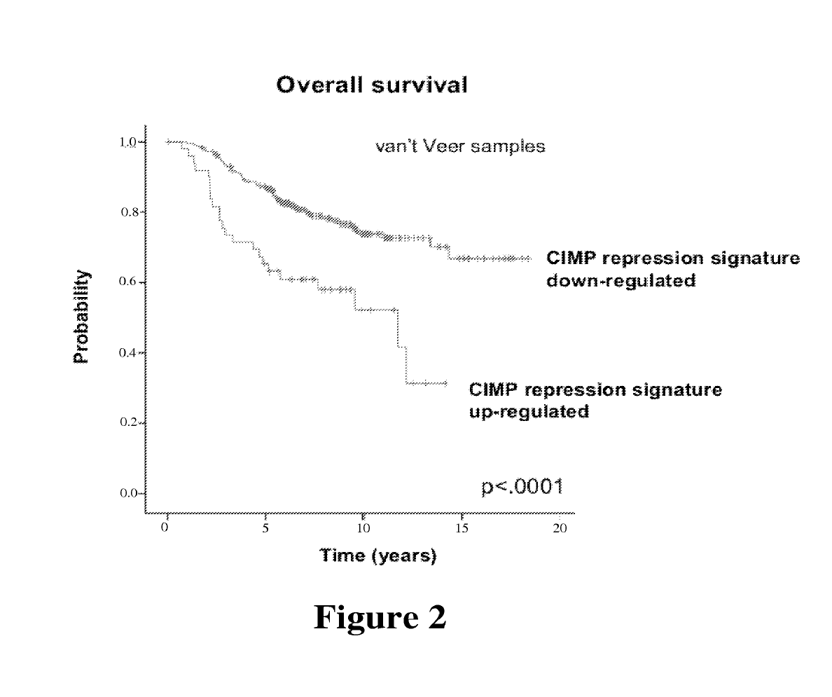

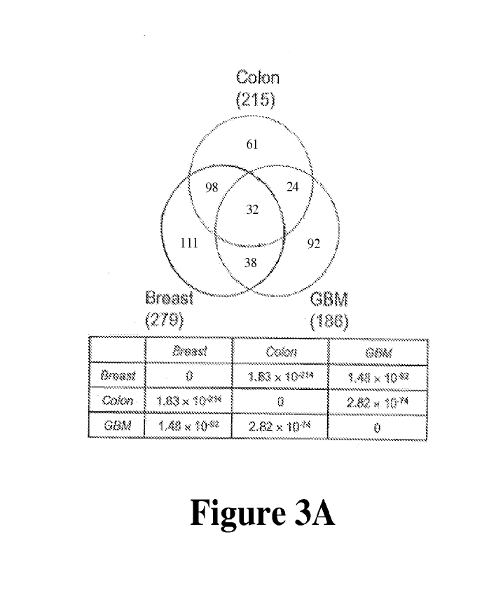

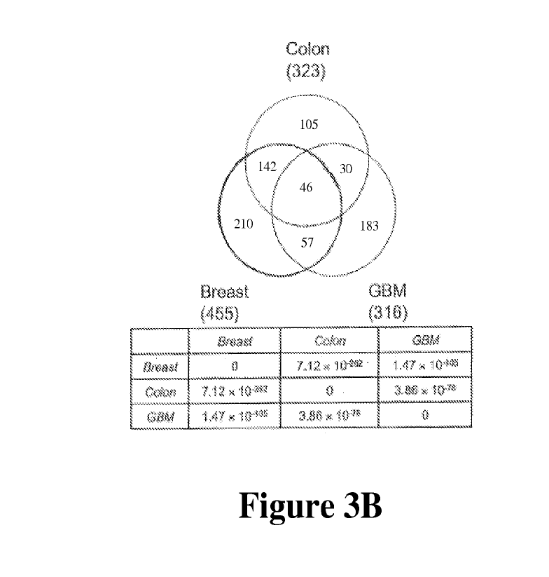

Although mRNA expression signatures have been developed to help predict the risk of metastatic disease in breast cancer patients, the genomic foundations for these differences in gene expression are incompletely understood (17, 39, 40). Few genetic changes have been shown to be causally related to these transcriptional differences. Since B-CIMP status affects metastatic risk, we wondered whether methylation helps account for the transcriptome diversity underlying common breast cancer prognostic expression signatures. To address this question, we performed concepts mapping analysis as previously described (41). Remarkably, the methylated and down-regulated genes comprising the transcriptomic footprint of B-CIMP (B-CIMP repression signature) were markedly enriched among the most differentially expressed genes defining prognosis in multiple breast cancer cohorts. Low expression of genes comprising the B-CIMP repression signature was seen in tumors that did not metastasize and high expression of the signature was seen in tumors which metastasized and/or resulted in poor survival (Tables 7 and 8). Importantly, we observed highly significant associations between B-CIMP genes and breast cancer relapse expression signatures from multiple independent data sets, confirming the validity of our findings. Using the van't Veer cohort, we demonstrated that the presence of the B-CIMP repression signature strongly predicted survival (FIG. 2). Again, breast cancers in which the CIMP repression signature was present had a significantly better survival than tumors lacking the signature. Furthermore, gene set enrichment analysis (GSEA) demonstrated a significant inverse correlation between B-CIMP repressed genes and genes up-regulated in highly metastatic tumors. Importantly, these findings indicate that epigenomic alterations associated with B-CIMP are a fundamental basis underlying many of the gene expression differences observed in currently used breast cancer prognostic signatures such as Mammaprint.

### Distinct Gene Sets Targeted by B-CIMP

To elucidate the differences in the methylation landscape between the two epigenomic subclasses, we mapped regions of the most significant methylation differences between CIMP+ and CIMP− tumors across the genome. Dense clusters of methylation density were apparent in the arms of a number of chromosomes. GSEA of the differentially methylated genes showed a highly significant enrichment for polycomb complex 2 (PRC2) targets (Table 3), the vast majority being CpG-island containing genes (42). B-CIMP genes were highly enriched in many PRC2 occupancy data sets, including those involving H3K27 methylation, SUZ12 and EZH2—in stem cells and in cancer cells. GSEA analysis using the Broad molecular signature database demonstrated that CIMP genes were most significantly enriched in polycomb (PcG) occupancy data sets, although other processes were also implicated, including EMT and Wnt signaling, which are known to play a role in metastasis (Tables 9 and 10) (43). It has been shown that the presence of a bivalent chromatin mark involving the key PcG mark, trimethylated H3K27, in stem cells may predispose specific genes to become hypermethylated and silenced in cancer and may be indicative of a contribution of stem cells to the derivation of specific cancers (44, 45). Perhaps this process is active in breast tumors of the B-CIMP subclass.

A further classifier set that allows a limited of number of genes to be used for prediction of metastatic risk in multiple cancer types, specifically breast and colon cancer and glioma was also developed. We compared the CIMP-associated loci from breast cancer, colon cancer, and glioma (publicly available from The Cancer Genome Atlas—http://cancergenome.nih.gov). CIMP-associated genes were defined for glioma and colon cancer using the same methodology as above and were consistent with previous data (1, 2). Colon CIMP genes were derived from MSKCC tumors (n=24) using hierarchical clustering and confirmed as described in Materials and Methods and in Weisenberger et al. (2006). All data sets were generated using the same Infinium HumanMethylation27 platform and were directly comparable. We first wished to determine whether CIMP selectively targeted PcG targets not only in breast cancer but in other malignancies as well. All methylated loci (beta-value FDR-corrected p-value<0.05) in the three tumor types were compared with previously generated global PcG target gene sets (46, 47) (Table 11). Highly significant overlap was observed between CIMP and PcG targets in breast, glioma, and colon cancer, potentially indicating that CIMP may employ similar processes across cancer types. Using the 33 most significant common predictors of CIMP, we generated a consensus signature for CIMP-positivity across these tumor types (Table 12). As CIMP imparts a favorable clinical prognosis in breast cancer, colon cancer (CIMP-hi, microsatellite unstable) (2, 48), and gliomas (1), this epigenomic signature can be used as an indicator of outcome across multiple human malignancies.

Our findings have several important implications for the understanding of breast and other cancer. First, we have definitively identified distinct epigenomic subtypes of breast cancer and documented the existence of a global CIMP in breast cancer. Aberrant hypermethylation of genes have been described in breast cancer previously (26, 49-51) and the methylation state of specific genes has been linked to outcome (52-54). However, the existence of a global CpG island methylator phenotype has remained elusive prior to our study. Our global approach robustly identifies B-CIMP as a characteristic of a subset of hormone-positive tumors. B-CIMP+ tumors demonstrated a lower propensity for metastasis and a better clinical outcome than B-CIMP− tumors. Interestingly, the association of better clinical outcome with CIMP+ tumors can be seen across multiple malignancies (breast, colon, and glioma) (2, 23). In these tumors, it may be that the epigenomic defects causing CIMP initially help promote neoplastic transformation but inactivate genes that may facilitate tumor aggressiveness in later stages of cancer progression. It is important to note, however, that the association of methylation at CIMP genes with good clinical outcome is not universally applicable to methylation at all genes. Methylation of specific candidate genes or groups of genes has been associated with poorer prognosis and these may has an effect on tumor aggressiveness independent of CIMP (27, 53, 55-57). Interestingly, genes such as these—including CDKN2A, PTPRD, and BRCA1—were not included among the B-CIMP loci.

Second, the genomic basis of prognostic transcriptional signatures is unclear. Importantly, our data, to our knowledge, for the first time demonstrate that aberrations in the DNA methylome explain many of the mRNA expression differences that underlie these signatures. The tight association of these changes with a genome-wide concerted hypermethylation phenotype and their enrichment for polycomb targets argues against the inactivation of these genes as being sporadic events. Rather, the B-CIMP phenotype is consistent with a global, systematic derangement in epigenetic regulation. Importantly, the methylome profiles we have derived and the associated CIMP repression signature provide a previously unknown mechanistic link between breast cancers with differing metastatic behavior and transcriptional signatures that predict metastatic relapse. It is important to note, although we show that methylation-associated gene silencing underlies many metastasis-associated gene expression changes, genetic changes are undoubtedly important as well. Indeed, mutations of a number of genes such as BRCA1, PTEN, and ERBB2 have been shown to be associated with an increased risk of metastasis (5, 58, 59). The relationship between these mutations and the B-CIMP phenotype is unclear and it is very likely that both genetic and epigenetic alterations contribute to the metastatic phenotype. Interestingly, BRCA1 has recently been shown to up-regulate DNMT1, which may help explain the association between BRCA1 mutation, basal-type tumors, and the lack of methylation we have observed in our study among hormone receptor-negative breast cancers (60). Future studies will be required to define any potential casual relationship between mutations and derangements in the epigenomic landscape.

### Materials and Methods

### Tumor Samples

Breast tumors (discovery set n=39, validation set n=132) from the Memorial Sloan-Kettering Cancer Center were obtained following patient consent and with institutional review board (IRB) approval. For the primary breast tumor data, tissues from primary breast cancers were obtained from therapeutic procedures performed as part of routine clinical management. Source DNAs or RNAs were extracted from frozen or paraffin-embedded primary tumors for the methylation and expression studies. Frozen samples were “snapfrozen” in liquid nitrogen and were stored at −80° C. Each sample was examined histologically with H&E-stained cryostat sections. Regions were

microdissected from the slides to provide a consistent tumor cell content of more than 70% in tissues used for analysis. Genomic DNA was extracted using the QIAamp DNA Mini kit or the QIAamp DNA FFPE Tissue kit (Qiagen) using the manufacturer's instructions. RNA was extracted using the Trizol (Invitrogen) according to the manufacturer's directions. Nucleic acid quality was determined using the Agilent 2100 Bioanalyzer. Nucleic acids from the discovery set were used for methylation and expression analysis as described below.

### Methylome Analysis Using the Infinium Platform

Genome-wide methylation analysis was performed using the Illumina Infinium HumanMethylation27 bead array. Bisulphite conversion of genomic DNA was done with the EZ DNA methylation Kit (Zymo Research) by following the manufacturer's protocol with modifications for the Illumina Infinium Methylation Assay. Briefly, one mg of genomic DNA was mixed with 5 μl of M-Dilution Buffer and incubated at 37° C. for 15 minutes and then mixed with 100 μl of CT Conversion Reagent prepared as instructed in the protocol. Bisulphite-converted DNA samples were desulphonated and purified. Bisulphite-converted samples were used for microarray or Epityper analysis. Bisulphite-converted genomic DNA was analyzed using the Infinium Human Methylation27 Beadchip Kit (Illumina, WG-311-1202) by the MSKCC Genomics Core. Processing of the array was per the manufacturer's protocol. Briefly, 4 μl of bisulphite-converted genomic DNA was denatured in 0.014N sodium hydroxide, neutralized and amplified with reagents from the kit and buffer for 20-24 hours at 37° C. Each sample was loaded onto a 12-sample array. After incubation at 48° C. for 16-20 hours, chips were washed with buffers provided in the kit and placed into a fluid flow-through station for primer-extension reaction. Chips were image-processed using Illumina's iScan scanner. Data were extracted using GenomeStudio software (Illumina). Methylation values for each CpG locus are expressed as a beta (?)-value, representing a continuous measurement from 0 (completely unmethylated) to 1 (completely methylated). This value is based on following calculation: ?-value=(signal intensity of methylation-detection probe)/(signal intensity of methylation-detection probe+signal intensity of non-methylation-detection probe). Methylation analysis controls included in vitro methylated DNA (positive control) (61) and human HCT 116 DKO DNA (DNA methyltransferase double knock-out cells (DNMT1 and DNMT3b) (62).

### Gene Expression Profiling

Methods for RNA extraction, labeling, and hybridization for DNA microarray analysis have been described previously (39). Briefly, complementary DNA was synthesized from total RNA using a T7 promoter-tagged dT primer. All gene expression analysis was carried out using the Affymetrix Human Genome U133A 2.0 microarray. Image acquisition was performed using an Affymetrix GeneChip scanner. Fluorescence intensities were background-corrected, mismatch-adjusted, normalized and summarized to yield log 2-transformed gene expression data.

### Data Analysis for Genomics

For expression analysis, the Affymetrix data were imported into the Partek Genomics Suite (Partek). Data were normalized, log-transformed, and median-centered for analysis. Analysis of variance (ANOVA) followed by false discovery correction (FDR) (63, 64) was used to identify genes that were differentially expressed between the CIMP groups. (See table 14) Hierarchical clustering was performed using wither Euclidean distance or Pearson correlation. SigClust significance as implemented in the R package sigclust was used as described in (33). For Gene Ontogeny analysis, functional analysis of gene lists was performed using the Database for Annotation, Visualization and Integrated Discovery (DAVID) (65, 66) and the PANTHER functional annotation classes. PANTHER categories with adjusted p-values (FDR-corrected with Benjamini-Hochberg)<0.05 were considered as significantly over-represented in our gene lists.

For methylation analysis, Illumina data was imported into Partek using custom software. Beta-values were logit-transformed and mean-centered prior to analysis. ANOVA with false discovery correction (FDR) (63, 64) was used to identify genes that were differentially methylated between the CIMP groups. Significant changes were defined as genes having an FDR-corrected p-value<0.05. Probes with an adjusted p-value below 0.05 were considered significantly differentially methylated between the two sets of tumors. The beta-value difference between the two groups was performed by first calculating the mean beta-value across each group and then calculating the difference between the mean beta-values for each probe. The hierarchical clustering of the methylation data was performed as above using the top 5% most variant probes across the samples (defined by standard deviation). K-means consensus clustering was performed using the R statistical package. The optimum cluster number was identified by varying K and evaluating the K-means output for significance of iterations. The top 5% of the most variably methylated probes between CIMP subgroups were retained, resulting in a 1359-gene by 39-sample matrix. Consensus clustering was performed on this matrix with k-means clustering (Kmax=9) using Euclidean distance and average linkage over 1000 resampling iterations with random restart (as implemented in GenePattern v3.2.3) (67). The consensus matrix for K=2 was imported into R statistical software (v.2.11.1) and the heatmap was visualized using the gplots and color Ramps packages in Bioconductor v2.6.

For identification of CIMP genes in colon cancer and glioblastoma, analysis was performed as follows. Methylation data for colon cancer were downloaded from The Cancer Genome Atlas (TCGA) data portal and imported into R statistical software. Hierarchical clustering was performed as described above with the breast cancer data using the top 5% most variant probes. Iterations using the top 3% to 20% did not significantly alter the clustering results. The cluster results were confirmed using the methylation b-values of the 5 gene panel described by Weisenberger et. al. to identify CIMP+ tumors in colorectal cancers (2). The cluster of samples that exhibited hypermethylation of these marker genes was selected as CIMP positive and used for further analyses. These corresponded to the cluster with high coordinate hypermethylation derived by hierarchical clustering. The glioblastoma CIMP genes were identified as described in (1). Datasets are deposited in the Gene Expression Omnibus and at www.cbio.mskcc.org. The Cancer Genome Atlas Project GBM cancer datasets are publically available at www.cancergenome.nih.gov.

### Concepts Module Mapping and Gene Set Enrichment Analysis

Concepts module mapping was performed as follows. The methylation signature identified from our analysis (table S1) was imported into Oncomine (http://www.oncomine.org) to search for associations with molecular concepts signatures derived from independent cancer profiling studies. We report statistically significant overlaps of our methylation gene signature with the top-ranking gene expression signatures of clinical outcome using percentile cutoffs (10%). Q-value is calculated as previously described (41).

Gene Set Enrichment Analysis (GSEA) was performed using GSEA (68) software v2.0.7 and MSigDB database v2.5 (68). We assessed the significance of the gene sets with the following parameters: number of permutations=1000 and permutation_type=phenotype with an FDR q-value cut-off of 25%. The most differentially expressed genes from statistically significant gene sets were identified using the ‘leading edge subset” that consists of genes with the most contribution to the enrichment score of a particular gene set. Enrichment of gene sets downloaded from the literature (as referenced in table S8) was analyzed together with the curated gene sets (MSigDB collection c2) or within each other.

### Quantitative DNA Methylation Analysis Using Mass Spectrometry

DNA methylation analysis was carried out using the Epityper system Sequenom. The EpiTYPER assay is a tool for the detection and quantitative analysis of DNA methylation using base-specific cleavage of bisulfite-treated DNA and matrix-assisted laser desorption/Ionization time-of-flight mass spectrometry (MALDI-TOF MS) (69). Specific PCR primers for bisulfate-converted DNA were designed using the EpiDesigner software (www.epidesigner.com), for the entire CpG island of the genes of interest. T7-promoter tags are added to the reverse primer to obtain a product that can be in vitro transcribed, and a 10-mer tag is added to the forward primer to balance the PCR conditions. For primer sequences, target chromosomal sequence, and Epityper-specific tags, see table S2. One mg of tumor DNA was subjected to bisulfate treatment using the EZ-96 DNA methylation kit, which results in the conversion of unmethylated cytosines into uracil, following the manufacturer's instructions (Zymo). PCR reactions were carried out in duplicate, for each of the 2 selected primer pairs, for a total of 4 replicates per sample. For each replicate, 1 ml of bisulfate-treated DNA was used as template for a 5 ml PCR reaction in a 384-well microtiter PCR plate, using 0.2 units of Kapa2G Fast HotStart DNA polymerase (Kapa Biosystems), 200 mM dNTPs, and 400 nM of each primer. Cycling conditions were: 94° C. for 15 minutes, 45 cycles of 94° C. for 20 seconds, 56° C. for 30 seconds, 72° C. for 1 minute, and 1 final cycle at 72° C. for 3 minutes. Unincorporated dNTPs were deactivated using 0.3 U of shrimp alkaline phosphatase (SAP) in 2 ml, at 37° C. for 20 minutes, followed by heat inactivation at 85° C. for 5 minutes. Two ml of SAP-treated reaction were transferred into a fresh 384-well PCR plate, and in vitro transcription and T cleavage were carried out in a single 5 ml reaction mix, using the MassCleave kit (Sequenom) containing 1×T7 polymerase buffer, 3 mM DTT, 0.24 ml of T Cleavage mix, 22 units of T7 RNA and DNA polymerase, and 0.09 mg/ml of RNAse A. The reaction was incubated at 37° C. for 3 h. After the addition of a cation exchange resin to remove residual salt from the reactions, 10 nl of Epityper reaction product were loaded onto a 384-element SpectroCHIP II array (Sequenom). SpectroCHIPs were analyzed using a Bruker Biflex III matrix-assisted laser desorption/ionization-time of flight (MALDI-TOF) mass spectrometer (SpectroREADER, Sequenom). Results were analyzed using the Epityper Analyzer software, and manually inspected for spectra quality and peak quantification. CIMP positivity was defined as a mean methylated allelic frequency of >50% or a two-fold increase over normal breast tissue and the CIMP-negative state.

### Statistical Analysis and CIMP-Repression Signature

The 295-sample set of Van't Veer microarray data (NKI295) was downloaded from Rosetta Inpharmatics website (17). Seventy genes out of 102 of our methylation signature were represented in NKI295 and were used to test for prognostic significance. An average expression value was calculated for our hypermethylated and downregulated in CIMP geneset across each sample of NKI295. (See Table 15) A two-way classifier was developed by separating the patients into two groups based on the average expression value of our methylation signature: CIMP repression signature up-regulated if the average expression value was >0 and CIMP repression signature down-regulated otherwise. Kaplan-Meier curves comparing survival of patient subgroups were generated using SPSS statistical software.

### REFERENCES

### All of the References Cited Herein are Incorporated Herein by Reference in their Entirety

1. H. Noushmehr, D. J. Weisenberger, K. Diefes, H. S. Phillips, K. Pujara, B, P. Berman, F. Pan, C. E. Pelloski, E. P. Sulman, K. P. Bhat, R. G. Verhaak, K. A. Hoadley, D. N. Hayes, C. M. Perou, H. K. Schmidt, L. Ding, R. K. Wilson, D. Van Den Berg, H. Shen, H. Bengtsson, P. Neuvial, L. M. Cope, J. Buckley, J. G. Heiman, S. B. Baylin, P. W. Laird & K. Aldape, Identification of a CpG island methylator phenotype that defines a distinct subgroup of glioma. Cancer Cell 17, 510-522 (2010).
2. D. J. Weisenberger, K. D. Siegmund, M. Campan, J. Young, T. I. Long, M. A. Faasse, G. H. Kang, M. Widschwendter, D. Weener, D. Buchanan, H. Koh, L. Simms, M. Barker, B. Leggett, J. Levine, M. Kim, A. J. French, S. N. Thibodeau, J. Jass, R. Haile & P. W. Laird, CpG island methylator phenotype underlies sporadic microsatellite instability and is tightly associated with BRAF mutation in colorectal cancer. Nat. Genet. 38, 787-793 (2006).
3. C. M. Perou, T. Sorlie, M. B. Eisen, M. van de Rijn, S. S. Jeffrey, C. A. Rees, J. R. Pollack, D. T. Ross, H. Johnsen, L. A. Akslen, O. Fluge, A. Pergamenschikov, C. Williams, S. X. Zhu, P. E. Lonning, A. L. Borresen-Dale, P. O. Brown & D. Botstein, Molecular portraits of human breast tumours. Nature 406, 747-752 (2000).
4. A. Jemal, R. Siegel, E. Ward, Y. Hao, J. Xu, T. Murray & M. J. Thun, Cancer statistics, 2008. CA Cancer J. Clin. 58, 71-96 (2008).
5. L. H. Saal, S. K. Gruvberger-Saal, C. Persson, K. Lovgren, M. Jumppanen, J. Staaf, G. Jonsson, M. M. Pires, M. Maurer, K. Holm, S. Koujak, S. Subramaniyam, J. Vallon-Christersson, H. Olsson, T. Su, L. Memeo, T. Ludwig, S. P. Ethier, M. Krogh, M. Szabolcs, V. V. Murty, J. Isola, H. Hibshoosh, R. Parsons & A. Borg, Recurrent gross mutations of the PTEN tumor suppressor gene in breast cancers with deficient DSB repair. Nat. Genet. 40, 102-107 (2008).
6. L. A. Carey, C. M. Perou, C. A. Livasy, L. G. Dressler, D. Cowan, K. Conway, G. Karaca, M. A. Troester, C. K. Tse, S. Edmiston, S. L. Deming, J. Geradts, M. C. Cheang, T. O, Nielsen, P. G. Moorman, H. S. Earp & R. C. Millikan, Race, breast cancer subtypes, and survival in the Carolina Breast Cancer Study. Jama. 295, 2492-2502 (2006).
7. B. Weigelt, Z. Hu, X. He, C. Livasy, L. A. Carey, M. G. Ewend, A. M. Glas, C. M. Perou & L. J. Van't Veer, Molecular portraits and 70-gene prognosis signature are preserved throughout the metastatic process of breast cancer. Cancer Res. 65, 9155-9158 (2005).
8. H. B. Muss, S. Woolf, D. Berry, C. Cirrincione, R. B. Weiss, D. Budman, W. C. Wood, I. C. Henderson, C. Hudis, E. Winer, H. Cohen, J. Wheeler & L. Norton, Adjuvant chemotherapy in older and younger women with lymph node-positive breast cancer. Jama. 293, 1073-1081 (2005).
9. M. J. Piccart-Gebhart, M. Procter, B. Leyland-Jones, A. Goldhirsch, M. Untch, I. Smith, L. Gianni, J. Baselga, R. Bell, C. Jackisch, D. Cameron, M. Dowsett, C. H. Barrios, G. Steger, C. S. Huang, M. Andersson, M. Inbar, M. Lichinitser, I. Lang, U. Nitz, H. Iwata, C. Thomssen, C. Lohrisch, T. M. Suter, J. Ruschoff, T. Suto, V. Greatorex, C. Ward, C. Straehle, E. McFadden, M. S. Dolci & R. D. Gelber, Trastuzumab after adjuvant chemotherapy in HER2-positive breast cancer. N. Engl. J. Med. 353, 1659-1672 (2005).
10. T. Sorlie, R. Tibshirani, J. Parker, T. Hastie, J. S. Marron, A. Nobel, S. Deng, H. Johnsen, R. Pesich, S. Geisler, J. Demeter, C. M. Perou, P. E. Lonning, P. O. Brown, A. L. Borresen-Dale & D. Botstein, Repeated observation of breast tumor subtypes in independent gene expression data sets. Proc. Natl. Acad. Sci. USA 100, 8418-8423 (2003).
11. O. I. Olopade & T. Grushko, Gene-expression profiles in hereditary breast cancer. N. Engl. J. Med. 344, 2028-2029 (2001).
12. K. Kalinsky, L. M. Jacks, A. Heguy, S. Patil, M. Drobnjak, U. K. Bhanot, C. V. Hedvat, T. A. Traina, D. Solit, W. Gerald & M. E. Moynahan, PIK3CA mutation associates with improved outcome in breast cancer. Clin. Cancer Res. 15, 5049-5059 (2009).
13. L. D. Wood, D. W. Parsons, S. Jones, J. Lin, T. Sjoblom, R. J. Leary, D. Shen, S. M. Boca, T. Barber, J. Ptak, N. Silliman, S. Szabo, Z. Derso, V. Ustyanksky, T. Nikolskaya, Y. Nikolsky, R. Karchin, P. A. Wilson, J. S. Kaminker, Z. Zhang, R. Croshaw, J. Willis, D. Dawson, M. Shipitsin, J. K. Willson, S. Sukumar, K. Polyak, B. H. Park, C. L. Pethiyagoda, P. V. Pant, D. G. Ballinger, A. B. Sparks, J. Hartigan, D. R. Smith, E. Suh, N. Papadopoulos, P. Buckhaults, S. D. Markowitz, G. Parmigiani, K. W. Kinzler, V. E. Velculescu & B. Vogelstein, The genomic landscapes of human breast and colorectal cancers. Science 318, 1108-1113 (2007).
14. T. Sjoblom, S. Jones, L. D. Wood, D. W. Parsons, J. Lin, T. D. Barber, D. Mandelker, R. J. Leary, J. Ptak, N. Silliman, S. Szabo, P. Buckhaults, C. Farrell, P. Meeh, S. D. Markowitz, J. Willis, D. Dawson, J. K. Willson, A. F. Gazdar, J. Hartigan, L. Wu, C. Liu, G. Parmigiani, B. H. Park, K. E. Bachman, N. Papadopoulos, B. Vogelstein, K. W. Kinzler & V. E. Velculescu, The consensus coding sequences of human breast and colorectal cancers. Science 314, 268-274 (2006).
15. L. Ding, M. J. Ellis, S. L1, D. E. Larson, K. Chen, J. W. Wallis, C. C. Harris, M. D. McLellan, R. S. Fulton, L. L. Fulton, R. M. Abbott, J. Hoog, D. J. Dooling, D.C. Koboldt, H. Schmidt, J. Kalicki, Q. Zhang, L. Chen, L. Lin, M. C. Wendl, J. F. McMichael, V. J. Magrini, L. Cook, S. D. McGrath, T. L. Vickery, E. Appelbaum, K. Deschryver, S. Davies, T. Guintoli, L. Lin, R. Crowder, Y. Tao, J. E. Snider, S. M. Smith, A. F. Dukes, G. E. Sanderson, C. S. Pohl, K. D. Delehaunty, C. C. Fronick, K. A. Pape, J. S. Reed, J. S. Robinson, J. S. Hodges, W. Schierding, N. D. Dees, D. Shen, D. P. Locke, M. E. Wiechert, J. M. Eldred, J. B. Peck, B. J. Oberkfell, J. T. Lolofie, F. Du, A. E. Hawkins, M. D. O'Laughlin, K. E. Bernard, M. Cunningham, G. Elliott, M. D. Mason, D. M. Thompson, Jr., J. L. Ivanovich, P. J. Goodfellow, C. M. Perou, G. M. Weinstock, R. Aft, M. Watson, T. J. Ley, R. K. Wilson & E. R. Mardis, Genome remodelling in a basal-like breast cancer metastasis and xenograft. Nature 464, 999-1005 (2010).
16. S. Paik, S. Shak, G. Tang, C. Kim, J. Baker, M. Cronin, F. L. Baehner, M. G. Walker, D. Watson, T. Park, W. Hiller, E. R. Fisher, D. L. Wickerham, J. Bryant & N. Wolmark, A multigene assay to predict recurrence of tamoxifen-treated, node-negative breast cancer. N. Engl. J. Med. 351, 2817-2826 (2004).
17. L. J. van't Veer, H. Dai, M. J. van de Vijver, Y. D. He, A. A. Hart, M. Mao, H. L. Peterse, K. van der Kooy, M. J. Marton, A. T. Witteveen, G. J. Schreiber, R. M. Kerkhoven, C. Roberts, P. S. Linsley, R. Bernards & S. H. Friend, Gene expression profiling predicts clinical outcome of breast cancer. Nature 415, 530-536 (2002).
18. A. J. Minn, G. P. Gupta, D. Padua, P. Bos, D. X. Nguyen, D. Nuyten, B. Kreike, Y. Zhang, Y. Wang, H. Ishwaran, J. A. Foekens, M. van de Vijver & J. Massague, Lung metastasis genes couple breast tumor size and metastatic spread. Proc. Natl. Acad. Sci. US A. 104, 6740-6745 (2007).
19. P. A. Jones & S. B. Baylin, The epigenomics of cancer. Cell 128, 683-692 (2007).
20. J. F. Costello, M. C. Fruhwald, D. J. Smiraglia, L. J. Rush, G. P. Robertson, X. Gao, F. A. Wright, J. D. Feramisco, P. Peltomaki, J. C. Lang, D. E. Schuller, L. Yu, C. D. Bloomfield, M. A. Caligiuri, A. Yates, R. Nishikawa, H. Su Huang, N. J. Petrelli, X. Zhang, M. S. O'Dorisio, W. A. Held, W. K. Cavenee & C. Plass, Aberrant CpG-island methylation has non-random and tumour-type-specific patterns. Nat. Genet. 24, 132-138 (2000).
21. A. P. Feinberg & B. Vogelstein, Hypomethylation distinguishes genes of some human cancers from their normal counterparts. Nature 301, 89-92 (1983).
22. J. G. Herman & S. B. Baylin, Gene silencing in cancer in association with promoter hypermethylation. N. Engl. J. Med. 349, 2042-2054 (2003).
23. M. Toyota, N. Ahuja, M. Ohe-Toyota, J. G. Herman, S. B. Baylin & J. P. Issa, CpG island methylator phenotype in colorectal cancer. Proc. Natl. Acad. Sci. USA. 96, 8681-8686 (1999).
24. Y. K. Bae, A. Brown, E. Garrett, D. Bornman, M. J. Fackler, S. Sukumar, J. G. Herman & E. Gabrielson, Hypermethylation in histologically distinct classes of breast cancer. Clin. Cancer Res. 10, 5998-6005 (2004).
25. J. S. Lee, M. J. Fackler, J. H. Lee, C. Choi, M. H. Park, J. H. Yoon, Z. Zhang & S. Sukumar, Basal-like breast cancer displays distinct patterns of promoter methylation. Cancer Biol. Ther. 9 (2010).
26. P. Novak, T. Jensen, M. M. Oshiro, G. S. Watts, C. J. Kim & B. W. Futscher, Agglomerative epigenetic aberrations are a common event in human breast cancer. Cancer Res. 68, 8616-8625 (2008).
27. T. A. Chan, S. Glockner, J. M. Yi, W. Chen, L. Van Neste, L. Cope, J. G. Herman, V. Velculescu, K. E. Schuebel, N. Ahuja & S. B. Baylin, Convergence of mutation and epigenetic alterations identifies common genes in cancer that predict for poor prognosis. PLoS Med. 5, e114 (2008).
28.1. Van der Auwera, C. Bovie, C. Svensson, X. B. Trinh, R. Limame, P. van Dam, S. J. van Laere, E. A. van Marck, L. Y. Dirix & P. B. Vermeulen, Quantitative methylation profiling in tumor and matched morphologically normal tissues from breast cancer patients. BMC Cancer 10, 97.
29. M. M. Gaudet, M. Campan, J. D. Figueroa, X. R. Yang, J. Lissowska, B. Peplonska, L. A. Brinton, D. L. Rimm, P. W. Laird, M. Garcia-Closas & M. E. Sherman, DNA hypermethylation of ESR1 and PGR in breast cancer: pathologic and epidemiologic associations. Cancer Epidemiol. Biomarkers Prev. 18, 3036-3043 (2009).
30. M. Campan, D. J. Weisenberger & P. W. Laird, DNA methylation profiles of female steroid hormone-driven human malignancies. Curr. Top. Microbiol. Immunol. 310, 141-178 (2006).
31. M. Ehrlich, C. B. Woods, M. C. Yu, L. Dubeau, F. Yang, M. Campan, D. J. Weisenberger, T. Long, B. Youn, E. S. Fiala & P. W. Laird, Quantitative analysis of associations between DNA hypermethylation, hypomethylation, and DNMT RNA levels in ovarian tumors. Oncogene 25, 2636-2645 (2006).
32. M. R. Estecio, P. S. Yan, A. E. Ibrahim, C. S. Tellez, L. Shen, T. H. Huang & J. P. Issa, High-throughput methylation profiling by MCA coupled to CpG island microarray. Genome Res. 17, 1529-1536 (2007).
33. Y. Liu, D. N. Hayes, A. Nobel & J. Marron, Statistical significance of clustering for high dimensional low sample size data. Journal of the American Statistical Association 103, 1281-1293 (2008).
34. M. Ehrich, M. R. Nelson, P. Stanssens, M. Zabeau, T. Liloglou, G. Xinarianos, C. R. Cantor, J. K. Field & D. van den Boom, Quantitative high-throughput analysis of DNA methylation patterns by base-specific cleavage and mass spectrometry. Proc. Natl. Acad. Sci. USA. 102, 15785-15790, doi:0507816102
35. Y. L. Choi, M. Bocanegra, M. J. Kwon, Y. K. Shin, S. J. Nam, J. H. Yang, J. Kao, A. K. Godwin & J. R. Pollack, LYN is a mediator of epithelial-mesenchymal transition and a target of dasatinib in breast cancer. Cancer Res. 70, 2296-2306.
36. W. Tapper, V. Hammond, S. Gerty, S. Ennis, P. Simmonds, A. Collins & D. Eccles, The influence of genetic variation in 30 selected genes on the clinical characteristics of early onset breast cancer. Breast Cancer Res. 10, R108 (2008).
37. T. Milovanovic, K. Planutis, A. Nguyen, J. L. Marsh, F. Lin, C. Hope & R. F. Holcombe, Expression of Wnt genes and frizzled 1 and 2 receptors in normal breast epithelium and infiltrating breast carcinoma. Int. J. Oncol. 25, 1337-1342 (2004).
38. P. Papageorgis, A. W. Lambert, S. Ozturk, F. Gao, H. Pan, U. Manne, Y. O. Alekseyev, A. Thiagalingam, H. M. Abdolmaleky, M. Lenburg & S. Thiagalingam, Smad signaling is required to maintain epigenetic silencing during breast cancer progression. Cancer Res. 70, 968-978.
39. A. J. Minn, G. P. Gupta, P. M. Siegel, P. D. Bos, W. Shu, D. D. Giri, A. Viale, A. B. Olshen, W. L. Gerald & J. Massague, Genes that mediate breast cancer metastasis to lung. Nature 436, 518-524 (2005).
40. D. X. Nguyen & J. Massague, Genetic determinants of cancer metastasis. Nat. Rev. Genet. 8, 341-352 (2007).
41. D. R. Rhodes, S. Kalyana-Sundaram, S. A. Tomlins, V. Mahavisno, N. Kasper, R. Varambally, T. R. Barrette, D. Ghosh, S. Varambally & A. M. Chinnaiyan, Molecular concepts analysis links tumors, pathways, mechanisms, and drugs. Neoplasia 9, 443-454 (2007).
42. M. Ku, R. P. Koche, E. Rheinbay, E. M. Mendenhall, M. Endoh, T. S. Mikkelsen, A. Presser, C. Nusbaum, X. Xie, A. S. Chi, M. Adli, S. Kasif, L. M. Ptaszek, C. A. Cowan, E. S. Lander, H. Koseki & B. E. Bernstein, Genomewide analysis of PRC1 and PRC2 occupancy identifies two classes of bivalent domains. PLoS Genet. 4, e1000242, doi:10.1371/journal.pgen.1000242 (2008).
43. A. Subramanian, P. Tamayo, V. K. Mootha, S. Mukherjee, B. L. Ebert, M. A. Gillette, A. Paulovich, S. L. Pomeroy, T. R. Golub, E. S. Lander & J. P. Mesirov, Gene set enrichment analysis: a knowledge-based approach for interpreting genome-wide expression profiles. Proc. Natl. Acad. Sci. USA. 102, 15545-15550 (2005).
44. J. E. Ohm, K. M. McGarvey, X. Yu, L. Cheng, K. E. Schuebel, L. Cope, H. P. Mohammad, W. Chen, V. C. Daniel, W. Yu, D. M. Berman, T. Jenuwein, K. Pruitt, S. J. Sharkis, D. N. Watkins, J. G. Herman & S. B. Baylin, A stem cell-like chromatin pattern may predispose tumor suppressor genes to DNA hypermethylation and heritable silencing. Nat. Genet. 39, 237-242 (2007).
45. Y. Schlesinger, R. Straussman, I. Keshet, S. Farkash, M. Hecht, J. Zimmerman, E. Eden, Z. Yakhini, E Ben-Shushan, B. E. Reubinoff, Y. Bergman, I. Simon & H. Cedar, Polycomb-mediated methylation on Lys27 of histone H3 pre-marks genes for de novo methylation in cancer. Nat. Genet. 39, 232-236 (2007).
46. T. I. Lee, R. G. Jenner, L. A. Boyer, M. G. Guenther, S. S. Levine, R. M. Kumar, B. Chevalier, S. E. Johnstone, M. F. Cole, K. Isono, H. Koseki, T. Fuchikami, K. Abe, H. L. Murray, J. P. Zucker, B. Yuan, G. W. Bell, E. Herbolsheimer, N. M. Hannett, K. Sun, D. T. Odom, A. P. Otte, T. L. Volkert, D. P. Bartel, D. A. Melton, D. K. Gifford, R. Jaenisch & R. A. Young, Control of developmental regulators by Polycomb in human embryonic stem cells. Cell 125, 301-313 (2006).
47. A. P. Bracken, N. Dietrich, D. Pasini, K. H. Hansen & K. Helin, Genome-wide mapping of Polycomb target genes unravels their roles in cell fate transitions. Genes Dev. 20, 1123-1136 (2006).
48. J. P. Issa, Colon cancer: it's CIN or CIMP. Clin. Cancer Res. 14, 5939-5940 (2008).
49. V. Birgisdottir, O. A. Stefansson, S. K. Bodvarsdottir, H. Hilmarsdottir, J. G. Jonasson & J. E. Eyfiord, Epigenetic silencing and deletion of the BRCA1 gene in sporadic breast cancer. Breast Cancer Res. 8, R38 (2006).
50. P. K. Lo, J. S. Lee, X. Liang, L. Han, T. Mori, M. J. Fackler, H. Sadik, P. Argani, T. K. Pandita & S. Sukumar, Epigenetic inactivation of the potential tumor suppressor gene FOXF1 in breast cancer. Cancer Res. 70, 6047-6058.
51. B. C. Christensen, K. T. Kelsey, S. Zheng, E. A. Houseman, C. J. Marsit, M. R. Wrensch, J. L. Wiemels, H. H. Nelson, M. R. Karagas, L. H. Kushi, M. L. Kwan & J. K. Wiencke, Breast cancer DNA methylation profiles are associated with tumor size and alcohol and folate intake. PLoS Genet. 6, e1001043.
52. P. A. Jones & S. B. Baylin, The fundamental role of epigenetic events in cancer. Nat. Rev. Genet. 3, 415-428 (2002).
53. M. V. Brock, C. M. Hooker, E. Ota-Machida, Y. Han, M. Guo, S. Ames, S. Glockner, S. Piantadosi, E. Gabrielson, G. Pridham, K. Pelosky, S. A. Belinsky, S.C. Yang, S. B. Baylin & J. G. Herman, DNA methylation markers and early recurrence in stage I lung cancer. N. Engl. J. Med. 358, 1118-1128 (2008).
54. J. W. Martens, A. L. Margossian, M. Schmitt, J. Foekens & N. Harbeck, DNA methylation as a biomarker in breast cancer. Future Oncol. 5, 1245-1256 (2009).
55.1. Nimmrich, A. M. Sieuwerts, M. E. Meijer-van Gelder, I. Schwope, J. Bolt-de Vries, N. Harbeck, T. Koenig, O. Hartmann, A. Kluth, D. Dietrich, V. Magdolen, H. Portengen, M. P. Look, J. G. Klijn, R. Lesche, M. Schmitt, S. Maier, J. A. Foekens & J. W. Martens, DNA hypermethylation of PITX2 is a marker of poor prognosis in untreated lymph node-negative hormone receptor-positive breast cancer patients. Breast Cancer Res. Treat. 111, 429-437 (2008).
56. G. Dominguez, J. Silva, J. M. Garcia, J. M. Silva, R. Rodriguez, C. Munoz, I. Chacon, R. Sanchez, J. Carballido, A. Colas, P. Espana & F. Bonilla, Prevalence of aberrant methylation of p14ARF over p16INK4a in some human primary tumors. Mutat. Res. 530, 9-17 (2003).
57. M. K. Wendt, A. N. Cooper & M. B. Dwinell, Epigenetic silencing of CXCL12 increases the metastatic potential of mammary carcinoma cells. Oncogene 27, 1461-1471 (2008).
58. D. J. Slamon, G. M. Clark, S. G. Wong, W. J. Levin, A. Ullrich & W. L. McGuire, Human breast cancer: correlation of relapse and survival with amplification of the HER-2/neu oncogene. Science 235, 177-182 (1987).
59. D. J. Slamon, B. Leyland-Jones, S. Shak, H. Fuchs, V. Paton, A. Bajamonde, T. Fleming, W. Eiermann, J. Wolter, M. Pegram, J. Baselga & L. Norton, Use of chemotherapy plus a monoclonal antibody against HER2 for metastatic breast cancer that overexpresses HER2. N. Engl. J. Med. 344, 783-792 (2001).
60. V. Shukla, X. Coumoul, T. Lahusen, R. H. Wang, X. Xu, A. Vassilopoulos, C. Xiao, M. H. Lee, Y. G. Man, M. Ouchi, T. Ouchi & C. X. Deng, BRCA1 affects global DNA methylation through regulation of DNMT1. Cell Res, doi:cr2010128 [pii]
61. M. Esteller, M. Toyota, M. Sanchez-Cespedes, G. Capella, M. A. Peinado, D. N. Watkins, J. P. Issa, D. Sidransky, S. B. Baylin & J. G. Herman, Inactivation of the DNA repair gene O6-methylguanine-DNA methyltransferase by promoter hypermethylation is associated with G to A mutations in K-ras in colorectal tumorigenesis. Cancer Res. 60, 2368-2371 (2000).
62. I. Rhee, K. E. Bachman, B. H. Park, K. W. Jair, R. W. Yen, K. E. Schuebel, H. Cui, A. P. Feinberg, C. Lengauer, K. W. Kinzler, S. B. Baylin & B. Vogelstein, DNMT1 and DNMT3b cooperate to silence genes in human cancer cells. Nature 416, 552-556 (2002).
63. J. D. Storey & R. Tibshirani, Statistical significance for genomewide studies. Proc. Natl. Acad. Sci. USA. 100, 9440-9445 (2003).
64. J. D. Storey, A direct approach to false discovery rates. Journal of the Royal Statistical Society 64, 479-498 (2002).
65. W. Huang da, B. T. Sherman & R. A. Lempicki, Systematic and integrative analysis of large gene lists using DAVID bioinformatics resources. Nat. Protoc. 4, 44-57 (2009).
66. G. Dennis, Jr., B. T. Sherman, D. A. Hosack, J. Yang, W. Gao, H. C. Lane & R. A. Lempicki, DAVID: Database for Annotation, Visualization, and Integrated Discovery. Genome Biol. 4, P3 (2003).
67. M. Reich, T. Liefeld, J. Gould, J. Lerner, P. Tamayo & J. P. Mesirov, GenePattern 2.0. Nat. Genet. 38, 500-501 (2006).
68. V. K. Mootha, C. M. Lindgren, K. F. Eriksson, A. Subramanian, S. Sihag, J. Lehar, P. Puigserver, E. Carlsson, M. Ridderstrale, E. Laurila, N. Houstis, M. J. Daly, N. Patterson, J. P. Mesirov, T. R. Golub, P. Tamayo, B. Spiegelman, E. S. Lander, J. N. Hirschhorn, D. Altshuler & L. C. Groop, PGC-1alpha-responsive genes involved in oxidative phosphorylation are coordinately downregulated in human diabetes. Nat. Genet. 34, 267-273 (2003).
69. S. J. Docherty, O. S. Davis, C. M. Haworth, R. Plomin & J. Mill, Bisulfite-based epityping on pooled genomic DNA provides an accurate estimate of average group DNA methylation. Epigenetics Chromatin 2, 3 (2009).

TABLE 1

| Gene | Gene Name | Probe Set |
|---|---|---|
| ALX4 | aristaless-like homeobox 4 | cg04988423, cg15817236 |
| CRABP1 | cellular retinoic acid binding protein 1 | cg11200929, cg16703647, |
| | | cg17133183, cg23337754 |
| ADAM23 | ADAM metallopeptidase domain 23 | cg16778809, cg26258845 |
| | preproprotein | |
| MOXD1 | monooxygenase; DBH-like 1 isoform 2 | cg07570142 |
| CHST2 | carbohydrate (N-acetylglucosamine-6-O) | cg00995327 |
| | sulfotransferase 2 | |
| FAM89A | hypothetical protein LOC375061 | cg00679738 |
| RNH1 | ribonuclease/angiogenin inhibitor | cg06417962 |
| B3GNT5 | beta-1;3-N-acetylglucosaminyltransferase | cg02238826, cg17701886 |
| | bGnT-5 | |
| KCNIP1 | Kv channel interacting protein 1 isoform 2 | cg08422599 |
| SLC16A12 | solute carrier family 16 (monocarboxylic | cg09186006, cg12005098 |
| | acid transporters); member 12 | |
| RUNX3 | runt-related transcription factor 3 isoform 2 | cg06377278 |
| LYN | v-yes-1 Yamaguchi sarcoma viral related | cg10539712 |
| | oncogene homolog | |
| PSAT1 | phosphoserine aminotransferase isoform 2 | cg25336579 |
| RASGRF2 | Ras protein-specific guanine nucleotide- | cg09952204 |
| | releasing factor 2 | |
| SOX8 | SRY (sex determining region Y)-box 8 | cg21530890 |
| ARHGEF7 | Rho guanine nucleotide exchange factor 7 | cg00557354 |
| | isoform a | |
| ADAM12 | ADAM metallopeptidase domain 12 isoform | cg13488201 |
| | 1 preproprotein | |
| PYGO1 | pygopus homolog 1 | cg19850348 |
| P2RY1 | purinergic receptor P2Y1 | cg22055427 |
| FLJ25477 | hypothetical protein LOC219287 isoform 2 | cg22029275 |
| FBN1 | fibrillin 1 | cg18671950 |
| PROX1 | prospero-related homeobox 1 | cg14019317 |
| FOXL2 | forkhead box L2 | cg14312526 |
| KCNJ2 | potassium inwardly-rectifying channel J2 | cg25724441 |
| SMOC1 | secreted modular calcium-binding protein 1 | cg15239123 |
| MCF2L2 | Rho family guanine-nucleotide exchange | cg27239157 |
| | factor | |
| BMP3 | bone morphogenetic protein 3 (osteogenic) | cg01049530 |
| | precursor | |
| TRIM29 | tripartite motif protein TRIM29 isoform beta | cg13625403 |
| GRIK1 | glutamate receptor; ionotropic; kainate 1 | cg21816539 |
| | isoform 1 precursor | |
| ALK | anaplastic lymphoma kinase Ki-1 | cg18277754 |
| C2orf32 | chromosome 2 open reading frame 32 | cg07080358 |
| VIM | vimentin | cg12874092 |
| AKAP12 | A-kinase anchor protein 12 isoform 1 | cg01555431 |
| EIF5A2 | eIF-5A2 protein | cg10541755 |
| DZIP1 | DAZ interacting protein 1 isoform 2 | cg04101379 |
| FLJ34922 | hypothetical protein FLJ34922 | cg18108623 |
| TMEM22 | transmembrane protein 22 | cg15980408 |
| LBX1 | transcription factor LBX1 | cg03285457 |
| GJA7 | gap junction protein; alpha 7; 45kDa | cg19586576 |
| | (connexin 45) | |
| HAAO | 3-hydroxyanthranilate 3;4-dioxygenase | cg01561916 |
| KLK10 | kallikrein 10 precursor | cg08818784 |
| ZAR1 | zygote arrest 1 | cg18342279 |
| DPYSL5 | dihydropyrimidinase-like 5 | cg26195812 |
| SLIT2 | slit homolog 2 | cg18972811 |
| RGS17 | regulator of G-protein signalling 17 | cg24317255 |
| KIAA1822 | KIAA1822 protein | cg02867079 |
| PTGFR | prostaglandin F receptor | cg03495868 |
| FBN2 | fibrillin 2 precursor | cg25084878 |
| ST6GALNA | ST6 (alpha-N-acetyl-neuraminyl-2;3-beta- | cg12601757 |
| C3 | galactosyl-1; 3)-N-acetylgalactosaminide | |
| | alpha-2;6-sialyltransferase 3 | |
| VAX1 | ventral anterior homeobox 1 | cg00263760 |
| GPR83 | G protein-coupled receptor 83 | cg27634151 |
| TBX2 | T-box 2 | cg13274713 |
| SIX3 | sine oculis homeobox homolog 3 | cg13163729 |
| ACADL | acyl-Coenzyme A dehydrogenase; long | cg09068528 |
| | chain precursor | |
| ASRGL1 | asparaginase-like 1 protein | cg13799206 |

Evaluation of the extent of CIMP for a given gene can be determined using variations of bisulfite sequencing. Methylation in CpG islands occurs on cytosine bases within the sequences. Bisulfite conversion of the nucleic acid converts unmethylated cytosines to uracil, and methylated cytosines to unmethylated cytosines. Thus, sequencing of the bisulfite conversion product and comparison with a reference sequence for the gene identifies the bases that were been methylated in the sample sequences. This type of procedure can be done using any type of assay platform that can distinguish between sequences containing Cs and sequences containing Us. This includes amplification of the relevant region and complete sequencing, high stringency hybridization assays that detect binding, high stringency amplification where the primer overlaps with the CpG island and amplifies only in the absence or presence of methylation, and similar techniques. One particular technique makes use of an Illumina Human Methylation27 beadarray, or a scaled down variant in which the probe sets used are those that provide information concerning genes methylated in IDC breast cancers with metastatic potential. This technique looks at 2 CpG sites per CpG island, although more sites would be evaluated in a more focused assay. See also US Patent Publication No. 2010/0209906 relating to detection of methylation in colon cancer.

Lengthy table referenced here
US20140113286A1-20140424-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20140113286A1). An electronic copy of the table will also be available from the USPTO upon request and payment of the fee set forth in 37 CFR 1.19(b)(3).

1. A method for assessing risk of metastasis in a cancer patient identified as having breast cancer, colon cancer or glioma, comprising the steps of:
(a) obtaining a sample of tumor tissue from the patient;
(b) evaluating the sample for hypermethylation of a plurality of genes, and
(c) based on the evaluation of step (b) determining whether and/or to what extent the patient is at risk of cancer metastasis,
wherein the plurality of genes includes at least three genes selected from the group consisting of: ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1.

2. The method of claim 1, wherein the number of genes evaluated is from 3 to 20 genes selected from the group consisting of ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1.

3. The method of claim 2, wherein the number of genes evaluated is from 3 to 10 genes selected from the group consisting of ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1.

4. The method of any claim 1, wherein the patient is identified as having breast cancer.

5. The method of claim 4, wherein the plurality of genes includes at least three genes selected from the group consisting RASGF2, ARHGEF7, FBN1, SOX8, CRABP1, FOXL2, and ALX4.

6. The method of claim 4, wherein the sample is evaluated for the genes RASGF2, ARHGEF7, FBN1, SOX8, CRABP1, FOXL2, and ALX4.

7. The method of claim 4, wherein at least 50 genes are evaluated.

8. The method of claim 4, wherein the genes evaluated include at least three genes selected from the group consisting of ALX4, CRABP1, ADAM23, MOXD1, CHST2, FAM89A, RNH1, B3GNT5, KCNIP 1, and SLC16A12.

9. The method of claim 4, wherein the genes evaluated include at least three genes selected from the group consisting of ALX4, CRABP1, ADAM23, MOXD1, CHST2, FAM89A, RNH1, and B3GNT5.

9. The method of claim 3, wherein the genes evaluated include at least three genes selected from the group consisting of ALX4, CRABP1, ADAM23, MOXD1, and CHST2.

10. The method of claim 3, wherein the genes evaluated include at least ALX4, CRABP1, and ADAM23.

11. The method of claim 1, wherein the genes evaluated include ADAM12, ALX4 and FOXL2.

12. The method according to claim 11, further comprising the step of evaluating at least one additional gene selected from the group consisting of ACOT12, ACTA1, AOX1, C1orf76, CD8A, DES, DMN, DMT1, DYPSL4, EYA4, FLJ14834, GCM2, GSH2, LOC112937, LOC389112937, LOC399458, MAL, MEGF10, MGC26856, NEUROG1, PDPN, RPL39L, SFRP2, SLC13A5, SYT6, TFP12, THSD3, TLR2 and TP73.

13. The method of claim 12, wherein the genes evaluated are ADAM12, ALX4, FOX12, ACOT12, ACTA1, AOX1, C1orf76, CD8A, DES, DMN, DMT1, DYPSL4, EYA4, FLJ14834, GCM2, GSH2, LOC112937, LOC389112937, LOC399458, MAL, MEGF10, MGC26856, NEUROG1, PDPN, RPL39L, SFRP2, SLC13A5, SYT6, TFP12, THSD3, TLR2 and TP73.

14. A kit consisting essentially of materials for the evaluation of metastasis risk in a cancer patient, said kit includes reagents for determination of the extent of methylation of a plurality of genes, wherein the plurality of genes includes at least three genes selected from the group consisting of: ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1.

15. The kit of claim 14, wherein the kit includes reagents for the detection of the extent of methylation of from 3 to 100 genes.

16. The kit of claim 14, wherein the kit includes reagents for the detection of the extent of methylation of from 3 to 50 genes.

17. The kit of claim 14, wherein the kit includes reagents for the detection of the extent of methylation of from 3 to 20 genes.

18. The kit of claim 14, wherein the kit includes reagents for the detection of the extent of methylation of from 3 to 10 genes.

19. The kit of claim 15, wherein the plurality of genes includes at least three genes selected from the group consisting RASGF2, ARHGEF7, FBN1, SOX8, CRABP1, FOXL2, and ALX4.

20. The kit of claim 19, wherein the kit includes reagents for detection of methylation for the genes RASGF2, ARHGEF7, FBN1, SOX8, CRABP1, FOXL2, and ALX4.

21. The kit of claim 15, wherein the plurality of genes include at least three genes selected from the group consisting of ALX4, CRABP 1, ADAM23, MOXD1, CHST2, FAM89A, RNH1, B3GNT5, KCNIP1, and SLC16A12.

22. The kit of claim 21, wherein the genes evaluated include at least three genes selected from the group consisting of ALX4, CRABP1, ADAM23, MOXD1, CHST2, FAM89A, RNH1, and B3GNT5.

23. The kit of claim 21, wherein the genes evaluated include at least three genes selected from the group consisting of ALX4, CRABP1, ADAM23, MOXD1, and CHST2.

24. The kit of claim 23, wherein the genes evaluated include at least ALX4, CRABP1, and ADAM23.

25. The kit of claim 15, wherein the genes evaluated include ADAM12, ALX4 and FOXL2.

26. The kit of claim 25, further comprising reagents for determination of the extent of methylation of one or more additional genes from the group consisting of ACOT12, ACTA1, AOX1, C1orf76, CD8A, DES, DMN, DMT1, DYPSL4, EYA4, FLJ14834, GCM2, GSH2, LOC112937, LOC389112937, LOC399458, MAL, MEGF10, MGC26856, NEUROG1, PDPN, RPL39L, SFRP2, SLC13A5, SYT6, TFP12, THSD3, TLR2 and TP73.

27. The kit of claim 15, including reagents for determination of the extent of methylation of ADAM12, ALX4, FOX12, ACOT12, ACTA1, AOX1, C1orf76, CD8A, DES, DMN, DMT1, DYPSL4, EYA4, FLJ14834, GCM2, GSH2, LOC112937, LOC389112937, LOC399458, MAL, MEGF10, MGC26856, NEUROG1, PDPN, RPL39L, SFRP2, SLC13A5, SYT6, TFP12, THSD3, TLR2 and TP73.

28. A kit for determination of risk of breast cancer metastasis, comprising reagents for determination of the extent of methylation in 100 or fewer genes, wherein at least 50% of the genes have relevance to the determination of risk of breast cancer metastasis, and wherein the plurality of genes includes at least three genes selected from the group consisting of: ALX4, CRABP1, ADAM23, MOXD1, CHST2, FAM89A, RNH1, B3GNT5, KCNIP1, SLC16A12, RUNX3, LYN, PSAT1, RASGRF2, SOX8, ARHGEF7, ADAM12, PYGO1, P2RY1, FLJ25477, FBN1, PROX1, FOXL2, KCNJ2, SMOC1, MCF2L2, BMP3, TRIM29, GRIK1, ALK, C2orf32, VIM, AKAP12, EIF5A2, DZIP1, FLJ34922, TMEM22, LBX1, GJA7, HAAO, KLK10, ZAR1, DPYSL5, SLIT2, RGS17, KIAA1822, PTGFR, FBN2, ST6GALNAC3, VAX1, GPR83, TBX2, SIX3, ACADL, and ASRGL1.
